(19) United States
(12) Patent Application Publication
(10) Pub. No.: US 2012/0310074 A1
(43) Pub. Date: Dec. 6, 2012

(54) MEDICAL IMAGE PROCESSING APPARATUS, A MEDICAL IMAGING APPARATUS, AND A MEDICAL IMAGE PROCESSING PROGRAM

(75) Inventors: Kyohei Yamamori, Otawara-shi (JP); Tomohiro Kawasaki, Otawara-shi (JP); Satoshi Wakai, Nasushiobara-shi (JP); Tetsuya Yokota, Otawara-shi (JP); Yoshifumi Yamagata, Otawara-shi (JP); Kensuke Shinoda, Otawara-shi (JP)

(73) Assignees: TOSHIBA MEDICAL SYSTEMS CORPORATION, Otawara-shi (JP); KABUSHIKI KAISHA TOSHIBA, Tokyo (JP)

(21) Appl. No.: 13/501,254

(22) PCT Filed: Oct. 21, 2011
(86) PCT No.: PCT/JP11/05904

§ 371 Date: Apr. 11, 2012

(30) Foreign Application Priority Data
Oct. 25, 2010 (JP) 2010-238310

(51) Int. Cl. A61B 5/02; A61B 6/00

(52) U.S. Cl. 600/407

(57) ABSTRACT

A medical image processing apparatus includes a morphological identification part, a function calculator and a display processor. The morphological identification part identifies morphological information related to thickness of a heart muscle of a subject or thickness of surrounding sites thereof from medical image data obtained by capturing an image of the subject using a medical imaging apparatus. The function calculator determines cardiac function information related to movement of the heart muscle of the subject based on the medical image data. The display processor causes a display to display a combination of the identified morphological information and the calculated cardiac function information which is identified by color. The medical image processing apparatus can easily diagnose diseases.

### TECHNICAL FIELD

Embodiments of the present invention relate to a medical image processing apparatus, a medical imaging apparatus, and a medical image processing program.

### BACKGROUND ART

In diagnosing heart diseases such as ischemic heart disease and constrictive pericarditis, medical image data obtained by means of medical imaging apparatuses such as X-ray CT scanners or MRI apparatuses are used. For example, three-dimensional images showing the heart are sterically displayed, the function of the heart is displayed on a bullseye map, or information showing the function of the heart is displayed by stacking medical images.

Constrictive pericarditis is a disease that causes damage to the systolic and diastolic functions of the heart muscle as a result of pericardial thickening, conglutination between the epicardium and the heart muscle, etc. There is a tendency for calcification to be caused at sites of adhesion; therefore, calcified regions are verified by means of images and calcified epicardium is surgically removed. In diagnosing constrictive pericarditis, it is necessary to identify calcified sites affecting the systolic and diastolic functions of the heart muscle. For example, physicians verify calcified regions by referring to medical images such as X-ray CT images, ultrasonic images, or X-ray images. Moreover, physicians verify abnormalities in heart muscle movement by referring to bullseye maps.

### PRIOR ART DOCUMENTS

### Patent Document

[Patent Document 1] Japanese Unexamined Patent Application Publication 2009-18005

### SUMMARY OF THE INVENTION

### Problems to be Solved by the Invention

It is difficult to verify abnormalities in organ movement (for example, the heart muscle) by means of X-ray images. Organ movement abnormalities may be verified by means of diagrams such as bullseye maps; however, it is difficult to identify the sites affecting organ movement (for example, calcified regions). Therefore, it is necessary for physicians to diagnose diseases by referring to other information (images such as X-ray images and diagrams such as bullseye maps).

The embodiments of the present invention are intended to solve the above problems, with the object of the invention being to provide a medical image processing apparatus, a medical imaging apparatus, and a medical image processing program that can easily diagnose diseases.

### Means for Solving the Problems

The medical image processing apparatus according to embodiments of the present invention comprises a morphological identification means, a function calculation means, and a display processing means. The morphological identification means identifies morphological information related to thickness of a heart muscle of a subject or thickness of surrounding sites thereof from medical image data obtained by capturing an image of the subject using a medical imaging apparatus. The function calculation means calculates cardiac function information related to movement of the heart muscle of the subject based on the medical image data. The display processing means causes a display to display a combination of the identified morphological information and the calculated cardiac function information which is identified by color.

### BRIEF DESCRIPTION OF THE DRAWINGS

FIG. 1 is a block diagram showing the medical image processing apparatus according to the first embodiment.

FIG. 2 is a diagram schematically showing the heart.

FIG. 3A is a diagram showing a short-axis image of the left ventricle at the end diastole.

FIG. 3B is a diagram showing a short-axis image of the left ventricle at the end systole.

FIG. 4A is a diagram showing a short-axis image of the left ventricle at the end diastole.

FIG. 4B is a diagram showing a short-axis image of the left ventricle at the end systole.

FIG. 5 is a diagram explaining the generating method of a bullseye map and is a diagram showing concentric circles.

FIG. 6 is a diagram showing bullseye maps and color maps.

FIG. 7 is a diagram showing a three-dimensional image of the heart.

FIG. 8 is a flow chart showing actions by means of the medical image processing apparatus according to the first embodiment.

FIG. 9 is a block diagram showing the medical image processing apparatus according to the second embodiment

FIG. 10A is a diagram showing a short-axis image.

FIG. 10B is a diagram showing a long-axis image.

FIG. 10C is a diagram showing a color map.

FIG. 11 is a diagram showing a display example of short-axis images and long-axis images.

FIG. 12 is a flow chart showing actions by means of the medical image processing apparatus according to the second embodiment.

FIG. 13 is a block diagram showing the medical image processing apparatus according to the third embodiment.

FIG. 14 is a diagram schematically showing the color map.

FIG. 15 is a flow chart showing actions by means of the medical image processing apparatus according to the third embodiment.

### MODES FOR CARRYING OUT THE INVENTION

### First Embodiment

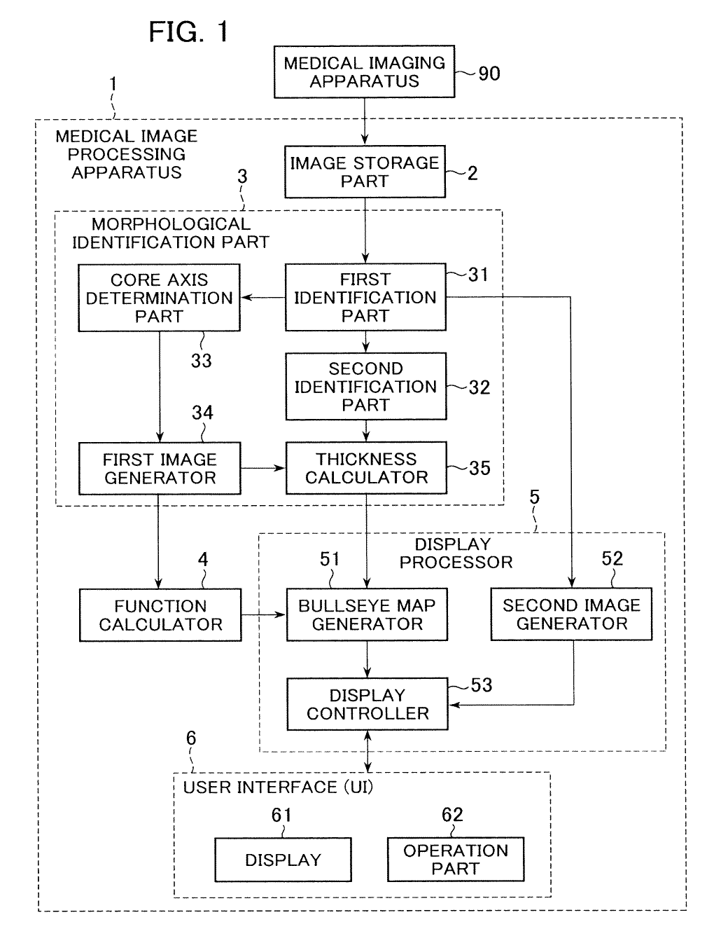

The medical image processing apparatus according to the first embodiment is described with reference to FIG. 1. For example, a medical imaging apparatus 90 is connected to a medical image processing apparatus 1 of the first embodiment.

### (Medical Imaging Apparatus 90)

Imaging apparatuses such as X-ray CT scanners or MRI apparatuses are used as the medical imaging apparatus 90. The medical imaging apparatus 90 has a capturing part and generates medical image data by capturing images of the region including an observation subject. For example, if the heart is the observation subject, the medical imaging apparatus 90 generates volume data showing the region including the heart by capturing images of the 3-dimensional region including the heart.

As an example, the medical imaging apparatus 90 captures continuous images of the heart to generate a plurality of volume data along time series. That is, the medical imaging apparatus 90 generates a plurality of volume data at different times of capturing images respectively. The medical imaging apparatus 90 outputs the plurality of volume data to the medical image processing apparatus 1.

So-called contrast imaging is also possible. In this case, the medical imaging apparatus 90 consecutively captures images of the heart of the subject to which a contrast agent is injected, to generate a plurality of volume data along time series.

The medical imaging apparatus 90 attaches time information to each volume data, the time information showing the time at which each volume data was generated. For example, electrocardiogram signals (ECG signals) of the subject are obtained using an electrocardiograph. The medical imaging apparatus 90 consecutively captures images of the heart of the subject, receives ECG signals from the electrocardiograph, and associates the ECG signals with the plurality of volume data. Accordingly, the time phase at which each volume data is generated is associated with each volume data. For example, the medical imaging apparatus 90 captures images of the heart over several heartbeats to generate a plurality of volume data over the several heartbeats.

### (Medical Image Processing Apparatus 1)

The medical image processing apparatus 1 comprises an image storage part 2, a morphological identification part 3, a function calculator 4, a display processor 5, and a user interface (UI) 6.

### (Image Storage Part 2)

The image storage part 2 stores the medical image data transmitted from the medical imaging apparatus 90. For example, the image storage part 2 stores the plurality of volume data showing the region including the heart.

The medical imaging apparatus 90 may not generate volume data but the medical image processing apparatus 1 may generate volume data. In such cases, the medical imaging apparatus 90 outputs a plurality of medical image data (for example, CT image data) to the medical image processing apparatus 1. The medical image processing apparatus 1 then generates volume data based on the plurality of medical image data.

### (Morphological Identification Part 3)

The morphological identification part 3 comprises a first identification part 31, a second identification part 32, a core axis determination part 33, a first image generator 34, and a thickness calculator 35. The morphological identification part 3 identifies shape of a heart based on the volume data and calculates morphological information showing shapes of sites in the heart having properties different from the heart. As an example of the shape of the sites having different properties, the morphological identification part 3 calculates thickness of calcified sites.

### (First Identification Part 31)

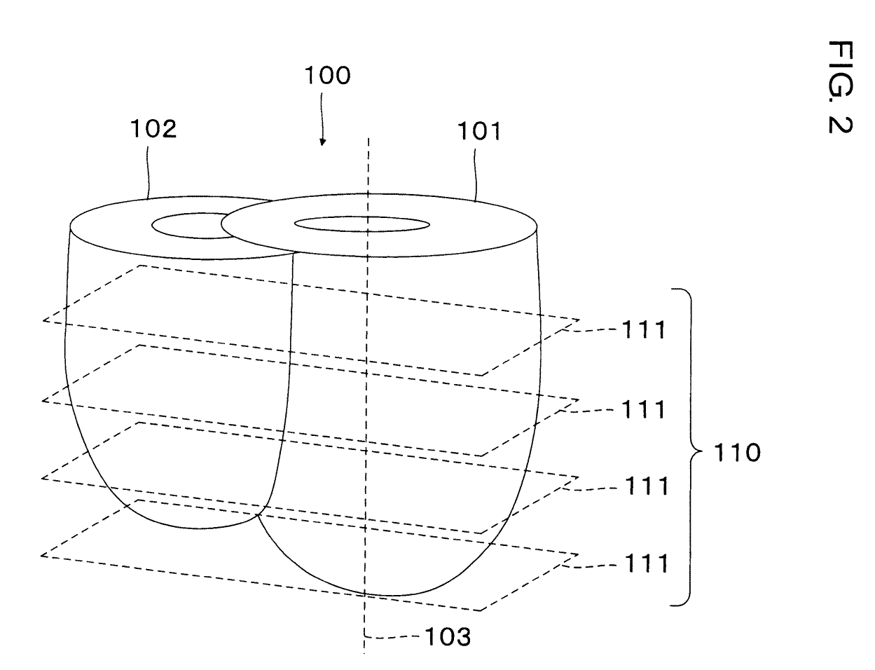

The first identification part 31 reads a plurality of volume data from the image storage part 2 and identifies the region of the heart from each volume data based on pixel values such as the CT value. For example, the first identification part 31 identifies the region of the heart from the volume data at the end diastole (End Diastole: ED), and identifies the region of the heart from the volume data at the end systole (End Systole: ES). That is, the first identification part 31 identifies the region of the heart at the end diastole and the region of the heart at the end systole. Alternatively, the first identification part may read the plurality of volume data generated within one heartbeat from the image storage part 2 and identify the region of the heart from the volume data generated at each time phase. An example of the heart identified by means of the first identification part 31 is shown in FIG. 2. FIG. 2 is a schematic diagram of the heart. For example, as shown in FIG. 2, the first identification part 31 identifies a heart 100 from the volume data. A left ventricle 101 and a right ventricle 102 are displayed in FIG. 2.

### (Second Identification Part 32)

The second identification part 32 identifies the calcified sites in the region of the heart from the volume data, upon receiving the volume data showing the region of the heart from the first identification part 31. For example, the second identification part 32 identifies calcified sites in the region of the heart based on pixel values such as the CT value. As an example, the second identification part 32 identifies the calcified sites in three-dimensional space, using a region growing method (region growing method). For example, the second identification part 32 may identify the calcified sites from the volume data at the end diastole or it may identify the calcified sites from the volume data at the end systole. That is, the second identification part 32 may identify the calcified sites at the end diastole or the calcified sites at the end systole. Alternatively, the second identification part 32 may identify the calcified sites from the volume data generated at each time phase in one heartbeat.

### (The Core Axis Determination Part 33)

Upon receiving the volume data showing the region of the heart from the first identification part 31, the core axis determination part 33 determines the core axis of the heart. As an example, the core axis determination part 33 determines the core axis of the left ventricle. For example, the core axis determination part 33 generates Multi Planar Reconstruction image (MPR) data by performing MPR processing on the volume data showing the heart. A display controller 53 causes a display 61 to display the MPR image based on the MPR image data. An operator uses an operation part 62 to specify the starting point and the ending point of the core axis on the MPR image displayed on the display 61. The coordinate information of the starting point and the ending point designated by the operator is output from the user interface (UI) 6 to the core axis determination part 33. The core axis determination part 33 defines a line passing through the starting point and the ending point as the core axis, upon receiving the coordinate information of the starting point and the coordinate information of the ending point.

The heart has a vertically long shape from the apex (the pointed part of the lower heart) to the base (the part from which blood vessels of the upper heart protrude). For example, the operator specifies the apex as the starting point and the base as the ending point, using the operation part 62. The core axis determination part 33 defines a line passing through the apex and the base as the core axis. For example, as shown in FIG. 2, the core axis determination part 33 determines a core axis 103 passing through the apex and the base and intersecting with the left ventricle 101.

For example, the core axis determination part 33 determines the core axis at the end diastole and the core axis at end systole. Alternatively, the core axis determination part 33 may calculate the core axis at each time phase within one heartbeat or it may calculate the core axis at any time phase.

### (First Image Generator 34)

The first image generator 34 generates image data in the short-axis cross-section orthogonally intersecting with the core axis (hereinafter, may be referred to as short-axis view data (short-axis view: SA)) by performing MPR processing on the volume data showing the region of the heart. For example, the first image generator 34 generates the SA view data at the end diastole based on the volume data at the end diastole. Moreover, the first image generator 34 generates the SA view data at the end systole based on the volume data at the end systole. Alternatively, the first image generator 34 may generate the SA view data at each time phase based on the volume data at each time phase within one heartbeat.

For example, as shown in FIG. 2, the first image generator 34 sets a plurality of short-axis cross-sections 111 at equal intervals in the region 110 between the starting point (the apex) and the ending point (the base) of the core axis 103. The first image generator 34 generates the SA view data in each short-axis cross-section 111 based on the volume data at the end diastole. As an example, the first image generator 34 sets 40 frames of the short-axis cross-sections 111 in the region 110 and generates 40 frames of the SA view data. For example, the operator specifies the number of short-axis cross-sections 111 and the interval length of the short-axis cross-sections 111 adjacent to each other, using the operation part 62. The information showing the number of short-axis cross-sections 111 and the interval length are output from the user interface (UI) 6 to the first image generator 34. The first image generator 34 generates the SA view data according to the number of short-axis cross-sections 111 and the interval length designated by the operator.

Similarly, the first image generator 34 generates SA view data in each short-axis cross-section 111 based on the volume data at the end systole. As an example, the first image generator 34 sets 40 frames of short-axis cross-sections 111 in the region 110 and generates 40 frames of SA view data.

### (Thickness Calculator 35)

The thickness calculator 35 calculates the thickness of the calcified sites in each short-axis cross-section, based on the calcified sites identified by means of the second identification part 32 and the plurality of SA view data generated by means of the first image generator 34. For example, the thickness calculator 35 calculates the thickness of the calcified sites in each short-axis cross-section at the end systole.

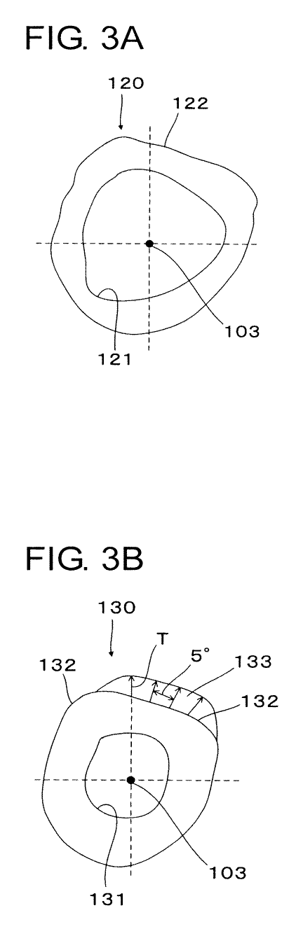

An explanation is provided regarding the processing that calculates the thickness of the calcified sites, with reference to FIG. 3A and FIG. 3B. FIG. 3A is a diagram showing a short-axis image of the left ventricle at the end diastole. FIG. 3B is a diagram showing a short-axis image of the left ventricle at the end systole. A short-axis image 120 is one frame image among the plurality of short-axis images at the end diastole. A short-axis image 130 is one frame image among the plurality of short-axis images at the end systole. The short-axis image 120 and the short-axis image 130 are images in the same short-axis cross-section. For example, the thickness calculator 35 calculates the thickness of the calcified sites based on the short-axis image 130 at the end systole. The thickness calculator 35 identifies the lateral wall 132 of the heart muscle shown in the short-axis image 130 based on the pixel value. The thickness calculator 35 may identify an inner wall 131 of the heart muscle. The thickness calculator 35 assumes the direction from the core axis 103 toward the lateral wall 132 to be a thickness direction to determine thickness T of a calcified site 133 on the lateral wall 132. For example, as shown in FIG. 3B, the thickness calculator 35 determines the thickness T of the calcified site 133 at 72 locations for each 5° interval centered on the rotation of the core axis 103. The thickness calculator 35 calculates thickness T of the calcified site 133 regarding each short-axis cross-section at the end systole. If 40 frames of the short-axis cross-sections 111 are set, the thickness calculator 35 calculates thickness T at 72 locations for one short-axis cross-section 111 regarding 40 frames of the short-axis cross-sections 111. Note that the 5° interval is an example and intervals at other angles may also be used to calculate the thickness T.

Alternatively, the thickness calculator 35 may determine thickness of the calcified sites in each short-axis cross-section at the end diastole. In such cases, the thickness calculator 35 identifies the lateral wall 122 of the heart muscle shown in the short-axis image 120 at the end diastole. The thickness calculator 35 may identify an inner wall 121 of the heart muscle. For example, the thickness calculator 35 determines thickness of the calcified site at 72 locations for each 5° interval regarding each short-axis cross-section at the end diastole.

### (Function Calculator 4)

The function calculator 4 determines the functional information showing functions of the heart (for example, the heart muscle movement) based on each SA view data generated by means of the first image generator 34. The function calculator 4 determines the difference in the lateral wall distance (wall motion) at the end diastole and the end systole as the functional information of the heart. Alternatively, the function calculator 4 may determine changes in the wall thickness of the heart muscle.

### (Differences in Lateral Wall Distance)

Cases of determining a difference in the lateral wall distance are set forth. The function calculator 4 determines the distance from the core axis to the lateral wall (the lateral wall distance) at the end diastole and determines the distance from the core axis to the lateral wall (the lateral wall distance) at the end systole. The function calculator 4 determines the difference between the lateral wall distance at the end diastole and the lateral wall distance at the end systole.

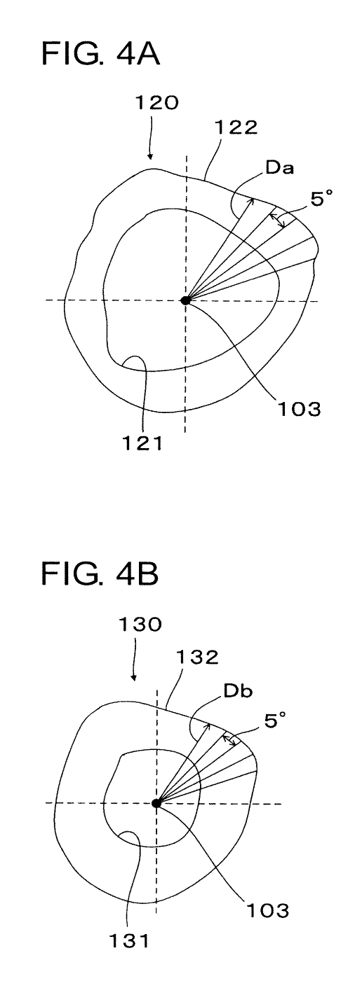

A detailed explanation is provided with reference to FIG. 4A and FIG. 4B. FIG. 4A is a diagram showing the short-axis image of the left ventricle at the end diastole. FIG. 4B is a diagram showing the short-axis image of the left ventricle at the end systole. The short-axis image 120 and the short-axis image 130 shown in FIG. 4A and FIG. 4B are images of the same short-axis cross-section. The function calculator 4 identifies the lateral wall 122 of the heart muscle shown in the short-axis image 120 at the end diastole based on the pixel value. The function calculator 4 may identify the inner wall 121 of the heart muscle. The function calculator 4 determines the distance Da from the core axis 103 to the lateral wall 122 (the lateral wall distance). For example, as shown in FIG. 4A, the function calculator 4 determines the distance Da at 72 locations for each 5° interval centered on the rotation of the core axis 103. The function calculator 4 determines the distance Da from the core axis 103 to the lateral wall 122 regarding each short-axis cross-section at the end diastole. If 40 frames of the short-axis cross-sections 111 are set, the function calculator 4 determines the distance Da at 72 locations for one short-axis cross-section 111 regarding 40 frames of the short-axis cross-sections 111. Note that the 5° interval is an example and intervals at other angles may also be used to determine the distance Da.

Moreover, the function calculator 4 identifies the lateral wall 132 of the heart muscle shown in the short-axis image 130 at the end systole based on the pixel value. The function calculator 4 may also identify the inner wall 131 of the heart muscle. The function calculator 4 determines the distance Db (the lateral wall distance) from the core axis 103 to the lateral wall 132. For example, as shown in FIG. 4B, the function calculator 4 determines the distance Db at 72 locations for each 5° interval centered on the rotation of the core axis 103. The function calculator 4 determines the distance Db from the core axis 103 to the lateral wall 132 regarding each short-axis cross-section at the end systole. If 40 frames of the short-axis cross-sections 111 are set, the function calculator 4 determines the distance Db at 72 locations for one short-axis cross-section 111 regarding 40 frames of the short-axis cross-sections 111. Note that the 5° interval is an example and intervals at other angles may also be used to determine the distance Da.

The function calculator 4 determines the difference between the distance Da at the end diastole (the lateral wall distance) and the distance Db at the end systole (the lateral wall distance) with regard to each location of each short-axis cross-section. For example, the function calculator 4 subtracts the distance Db at the end systole from the distance Da at the end diastole and refers to the value obtained as a result of the subtraction as the difference in the lateral wall distance. If 40 frames of the short-axis cross-sections 111 are set, the function calculator 4 determines the difference in the lateral wall distance at 72 locations for one short-axis cross-section 111 regarding 40 frames of the short-axis cross-sections 111. Accordingly, the difference in the lateral wall distance for 40 frames is determined at 72 locations for one short-axis cross-section 111.

### (Changes in the Wall Thickness)

An explanation of determining changes in wall thickness is provided. The function calculator 4 determines a distance between the lateral wall distance and the inner wall distance at the end diastole as the wall thickness. The function calculator 4 determines the distance Da from the core axis 103 to the lateral wall 122 (the lateral wall distance) at the end diastole. The function calculator 4 determines the distance from the core axis 103 to the inner wall 121 (the inner wall distance) at the end diastole. The function calculator 4 subtracts the inner wall distance from the lateral wall distance at the end diastole and refers to the value obtained as a result of the subtraction as the wall thickness. For example, the function calculator 4 determines the wall thickness at 72 locations for each 5° interval centered on the rotation of the core axis 103. The function calculator 4 determines the wall thickness in each short-axis cross-section at the end diastole. If 40 frames of the short-axis cross-sections 111 are set, the function calculator 4 determines the wall thickness at 72 locations for one short-axis cross-section 111 regarding 40 frames of the short-axis cross-sections 111. Note that the 5° interval is an example and intervals at other angles may also be used to calculate the wall thickness.

Moreover, the function calculator 4 determines the difference between the lateral wall distance and the inner wall distance at the end systole as the wall thickness. The function calculator 4 determines the distance Db from the core axis 103 to the lateral wall 132 (the lateral wall distance) at the end systole. The function calculator 4 determines the distance from the core axis 103 to the inner wall 131 (the inner wall distance) at the end systole. The function calculator 4 subtracts the inner wall distance from the lateral wall distance at the end systole and refers to the value obtained as a result of the subtraction as the wall thickness. For example, the function calculator 4 determines the wall thickness at 72 locations for each 5° interval centered on the rotation of the core axis 103. The function calculator 4 determines the wall thickness in each short-axis cross-section at the end systole. If 40 frames of the short-axis cross-sections 111 are set, the function calculator 4 determines the wall thickness at 72 locations for one short-axis cross-section 111 regarding 40 frames of the short-axis cross-sections 111. Note that the 5° interval is an example and intervals at other angles may also be used to calculate the wall thickness.

The function calculator 4 determines the difference between the wall thickness at the end diastole and the wall thickness at the end systole for each location at each short-axis cross-section. For example, the function calculator 4 subtracts the wall thickness at the end systole from the wall thickness at the end diastole and refers to the value obtained as a result of the subtraction as the difference in the wall thickness. The function calculator 4 divides the difference in the wall thickness by the wall thickness at the end systole and refers to the value obtained as a result of the division as a change in the wall thickness. For example, if 40 frames of the short-axis cross-sections 111 are set, the function calculator 4 determines changes in the wall thickness at 72 locations for one short-axis cross-section 111 regarding 40 frames of the short-axis cross-sections 111. Accordingly, changes in the wall thickness for 40 frames are determined at 72 locations for one short-axis cross-section 111.

### (Display Processor 5)

The display processor 5 comprises a bullseye map generator 51, a second image generator 52, and a display controller 53.

### (Bullseye Map Generator 51)

The bullseye map generator 51 generates a bullseye map based on morphological information calculated by means of the morphological identification part 3 and functional information calculated by means of the function calculator 4. For example, the bullseye map generator 51 generates a bullseye map based on the thickness of the calcified sites and the difference in the lateral wall distance. Alternatively, the bullseye map generator 51 may generate the bullseye map based on the thickness of the calcified sites and changes in the wall thickness.

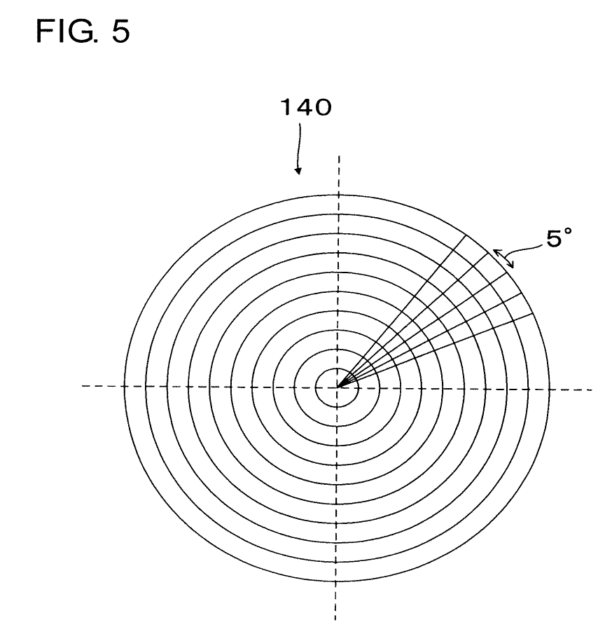

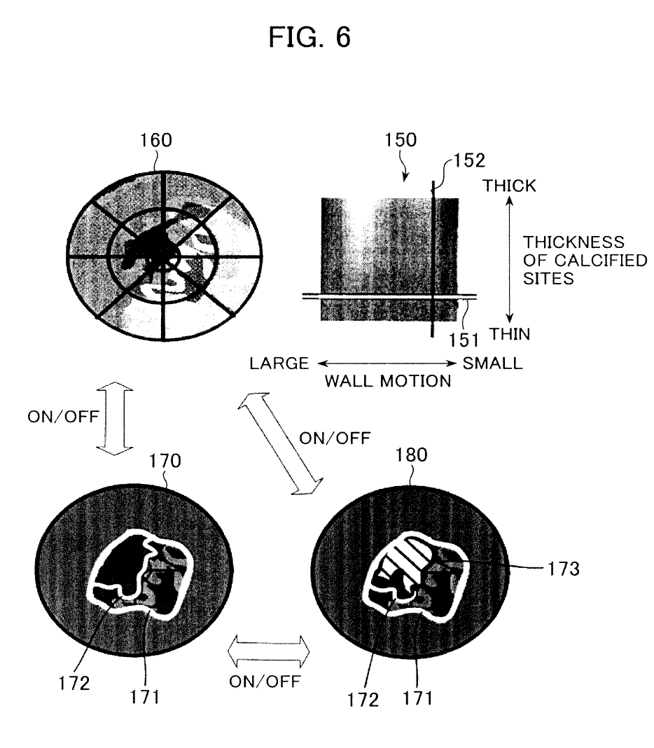

With reference to FIG. 5 and FIG. 6, an explanation of generating method of a bullseye map is provided. FIG. 5 is a diagram explaining the generating method of a bullseye map and is a diagram showing concentric circles. FIG. 6 is a diagram showing bullseye maps and color maps.

First, the bullseye map generator 51 determines the color corresponding to a combination of morphological information and functional information, using a two-dimensional color map having two axes. That is, the bullseye map generator 51 converts the combination of morphological information and functional information into a color. An example of the color map is shown in FIG. 6. For example, the bullseye map generator 51 uses the color map 150 shown in FIG. 6. In the color map 150, the horizontal axis corresponds to the difference in the lateral wall distance (or changes in the wall thickness) and the vertical axis corresponds to the thickness of the calcified sites. The color map 150 shows the distribution of, for example, the combination of color phase and color saturation. For example, the difference in the lateral wall distance (or changes in the wall thickness) corresponds to the color phase, while the thickness of the calcified sites corresponds to the color saturation. That is, the color map 150 defines the combination of color phase and color saturation corresponding to the combination of the difference in the lateral wall distance (or changes in the wall thickness) and the thickness of the calcified site. For example, colors are applied in the color map 150 such that the larger the difference in the lateral wall distance, the redder the color turns, while the smaller the difference in the lateral wall distance, the bluer the color turns. Moreover, colors are applied in the color map 150 such that the thicker the calcified sites, the higher the color saturation turns, while the thinner the calcified sites, the lower the color saturation turns. The color map 150 is prepared in advance and stored in a storage (not shown in the figures).

As an example, the bullseye map generator 51 determines the color corresponding to the combination of the thickness of the calcified site and the difference in the lateral wall distance, using the color map 150. Specifically, the bullseye map generator 51 determines the coordinate of the horizontal axis based on the difference in the lateral wall distance, calculates the coordinate of the vertical axis based on the thickness of the calcified sites, and identifies the color corresponding to the coordinate of the horizontal axis and the coordinate of the vertical axis from the color map 150. The bullseye map generator 51 may use the thickness of the calcified sites at the end systole or may use the thickness of the calcified sites at the end diastole, as the thickness of the calcified site.

The bullseye map generator 51 determines colors at locations in each short-axis cross-section. For example, the bullseye map generator 51 determines colors at 72 locations for one short-axis cross-section 111 regarding 40 frames of the short-axis cross-sections 111.

The bullseye map generator 51 generates the bullseye map using colors at respective locations in each short-axis cross-section 111. The bullseye map is shown in polar coordinate format. The angular direction (a direction) in the bullseye map is equivalent to the angular direction of the short-axis cross-section 111 shown in the polar coordinate, while the axis direction (r direction) in the bullseye map is equivalent to the core axis direction. Therefore, the bullseye map generator 51 allocates the color at each location (for example, at 72 locations) in the short-axis cross-section 111 at the apex to the inner most circle in the bullseye map, while allocating the color at each location (for example, at 72 locations) in the short-axis cross-section 111 at the base to the outermost circle in the bullseye map. That is, the bullseye map generator 51 plots the color at each location (for example, at 72 locations) in the short-axis cross-section 111 at the apex on the inner most circle of the bullseye map and plots the color at each location (for example, at 72 locations) in the short-axis cross-section 111 at the base on the outermost circle of the bullseye map. For example, as shown in FIG. 5, the bullseye map generator 51 sets the apex as the center of the circle 140 and sets the base as the outermost side of the circle 140, so as to plot colors of 40 frames on each concentric circle with regard to colors at 72 locations at the 5° interval for one frame of the short-axis cross-section 111.

The bullseye map generator 51 may generate the bullseye map using changes in the wall thickness. In such cases, the bullseye map generator 51 uses the color map 150 to determine colors corresponding to combinations of thickness of the calcified sites and changes in the wall thickness, so as to generate the bullseye map.

### (Second Image Generator 52)

Upon receiving volume data showing the region of the heart from the first identification part 31, the second image generator 52 generates three-dimensional image data sterically showing the heart by applying volume rendering on the volume data. For example, the second image generator 52 generates three-dimensional image data of the heart at the end systole based on the volume data at the end systole. The second image generator 52 may generate three-dimensional image data of the heart at the end diastole based on the volume data at the end diastole. The second image generator 52 may generate MPR image data at any cross-section by applying MPR processing on the volume data showing the region of the heart.

### (Display Controller 53)

The display controller 53 causes the display 61 to display the bullseye map generated by means of the bullseye map generator 51. The display controller 53 may cause the display 61 to display the three-dimensional image based on the three-dimensional image data generated by means of the second image generator 52. The display controller 53 may cause the display 61 to display the bullseye map and the three-dimensional image side by side.

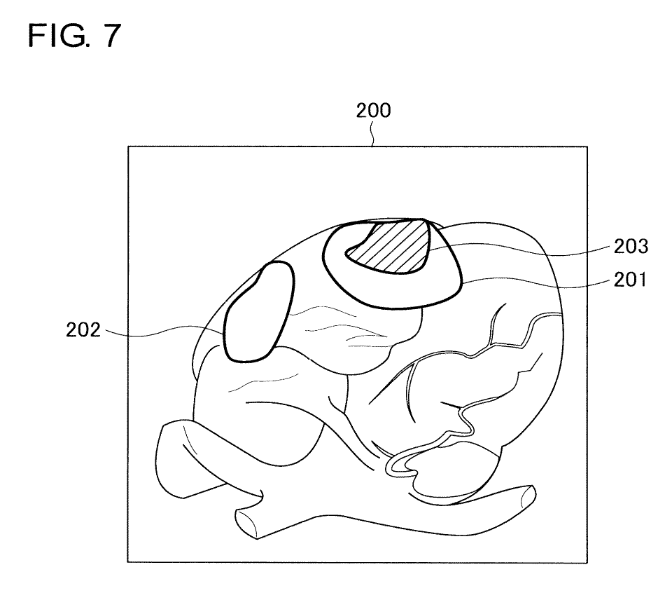

Display examples of the image are shown in FIG. 6 and FIG. 7. FIG. 7 is a diagram showing a three-dimensional image of the heart. As shown in FIG. 6, the display controller 53 causes the display 61 to display the bullseye map 160. The bullseye map 160 shows a distribution of colors corresponding to the combination of the difference in the lateral wall distance (or changes in the wall thickness) and the thickness of the calcified sites. Moreover, as shown in FIG. 7, the display controller 53 may cause the display 61 to display a three-dimensional image 200 of the heart. The display controller 53 may cause the display 61 to display the bullseye map 160 and the three-dimensional image 200 side by side.

The display controller 53 may also cause the display 61 to display a two-dimensional color map 150. For example, the display controller 53 cause the display 61 to display a first threshold bar 151 and a second threshold bar 152 to be superimposed on the color map 150. The first threshold bar 151 is used in order to set the first threshold with respect to morphological information (the thickness of the calcified sites). The second threshold bar 152 is used in order to set the second threshold with respect to functional information. The first threshold and the second threshold are values for restricting the display region of the bullseye map. The display controller 53 moves the first threshold bar 151 in the vertical axial direction and the second threshold bar 152 in the horizontal axial direction, upon receiving commands from the operator using the operation part 62. The operator specifies the first threshold with respect to the thickness of the calcified sites by operating the first threshold bar 151 using the operation part 62. Moreover, the operator specifies the second threshold with respect to the difference in the lateral wall distance (or changes in the wall thickness) by operating the second threshold bar 152 using the operation part 62. The display controller 53 restricts the display region of the bullseye map according to the first threshold and the second threshold.

For example, if the first threshold is set with respect to the thickness of the calcified sites, the display controller 53 causes the display 61 to display the region of the bullseye map in which the thickness of the calcified sites exceeds the first threshold. As shown in FIG. 6, the display controller 53 causes the display 61 to display a bullseye map 170 showing the region surrounded by a frame 171. The region surrounded by the frame 171 is the region in which the thickness of the calcified sites exceeds the first threshold. In other words, the region in which the thickness of the calcified sites is thinner than the first threshold is not displayed. Moreover, if the second threshold is set with respect to the difference in the lateral wall distance (or changes in the wall thickness), the display controller 53 may cause the display 61 to display a frame 172 surrounding the region of the bullseye map 170 in which the difference in the lateral wall distance (or changes in the wall thickness) is smaller than the second threshold.

If the first threshold and the second threshold are set, the display controller 53 may cause the display 61 to display as the critical region the region of the bullseye map in which the thickness of the calcified sites exceeds the first threshold and the difference in the lateral wall distance (or changes in the wall thickness) is smaller than the second threshold. For example, as shown in FIG. 6, the display controller 53 causes the display 61 to display a bullseye map 180 showing the region surrounded by the frame 171. The region surrounded by the frame 171 is the region in which the thickness of the calcified sites exceeds the first threshold. Furthermore, the display controller 53 causes the display 61 to display a region 173 (the region shown in hatching), in which the difference in the lateral wall distance (changes in the wall thickness) is smaller than the second threshold, as the critical region within the frame 171 of the bullseye map 180. In this way, the display controller 53 causes the display 61 to display the region in which the thickness of the calcified site exceeds the first threshold and the difference in the lateral wall distance (or changes in the wall thickness) is smaller than the second threshold so as to be distinguishable on the bullseye map.

In the three-dimensional image 200 shown in FIG. 7, the display controller 53 may cause the display 61 to display the region restricted by means of the first threshold and the second threshold so as to be distinguishable. For example, the display controller 53 causes the display 61 to display a region 201 corresponding to the region surrounded by the frame 171 in the bullseye map 170 so as to be distinguishable on the three-dimensional image 200. As an example, the display controller 53 causes the display 61 to display the region 201 in which the thickness of the calcified sites exceeds the first threshold by surrounding it with a frame or applying color in the three-dimensional image 200. Moreover, the display controller 53 may cause the display 61 to display a region 202 corresponding to the region surrounded by the frame 172 in the bullseye map 170 so as to be distinguishable in the three-dimensional image 200. As an example, the display controller 53 causes the display 61 to display the region 202 in which the difference in the lateral wall distance (or changes in the wall thickness) is smaller than the second threshold by surrounding it with the frame or applying color in the three-dimensional image 200. Moreover, the display controller 53 may cause the display 61 to display the region 203 corresponding to the region 173 defined as the critical region so as to be distinguishable in the three-dimensional image 200. As an example, the display controller 53 causes the display 61 to display the region 203 by surrounding it with the frame or applying color in the three-dimensional image 200.

### (User Interface (UI) 6)

The user interface (UI) 6 includes the display 61 and the operation part 62. The display 61 comprises a monitor such as a CRT and a liquid crystal display. The operation part 62 includes an input device such as a keyboard or a mouse.

The morphological identification part 3, the function calculator 4, and the display processor 5 may comprise a processing device (not shown in the figures) such as a CPU, a GPU, or an ASIC, respectively, as well as a storage device (not shown in the figures) such as a ROM, a RAM, or a HDD. Stored in the storage device are a morphological identification program for executing the function of the morphological identification part 3, a function calculation program for executing the function of the function calculator 4, and a display processing program for executing the function of the display processor 5. Included in the morphological identification program are a first identification program for executing the function of the first identification part 31, a second identification program for executing the function of the second identification part 32, a core axis determination program for executing the function of the core axis determination part 33, a first image generation program for executing the function of the first image generator 34, and a thickness calculation program for executing the function of the thickness calculator 35. Included in the display processing program are a bullseye map generation program for executing the function of the bullseye map generator 51, a second image generation program for executing the function of the second image generator 52, and a display control program for executing the function of the display controller 53. The function of each part is executed as a processing device such as a CPU executes each program stored in the storage device. Note that the morphological identification program, the function calculation program, and the display processing program indicate one example of “a medical image processing program” of the present invention.

### (Action)

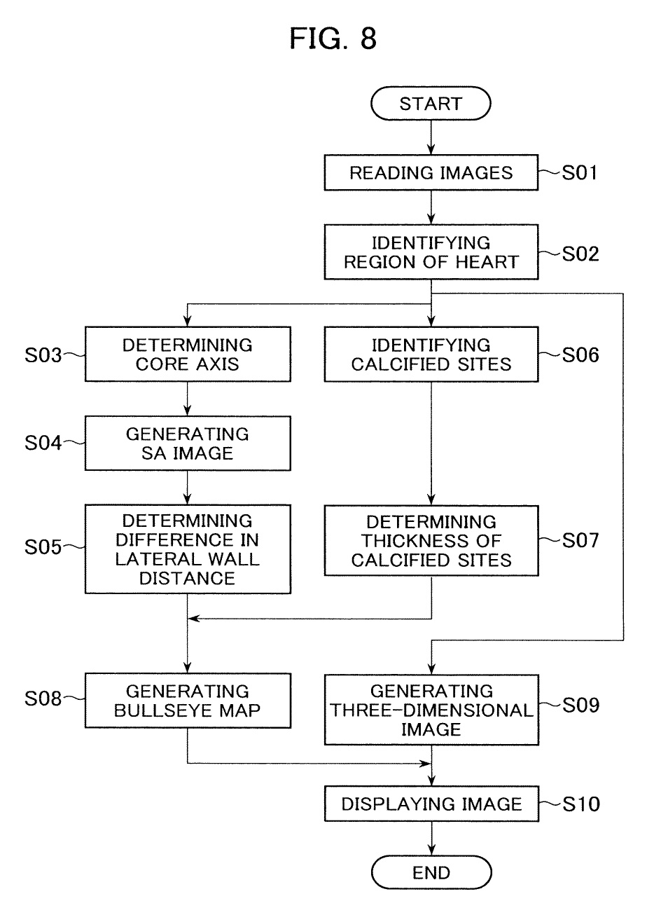

An explanation is provided regarding the action of the medical image processing apparatus 1 according to the first embodiment, with reference to the flow chart shown in FIG. 8.

### (Step S01)

The first identification part 31 reads the plurality of volume data from the image storage part 2.

### (Step S02)

The first identification part 31 identifies the region of the heart from each volume data based on pixel values such as the CT value. For example, the first identification part 31 identifies the region of the heart from the volume data at the end diastole and identifies the region of the heart from the volume data at the end systole.

### (Step S03)

The core axis determination part 33 determines the core axis of the heart upon receiving the volume data showing the region of the heart from the first identification part 31. For example, the operator specifies the apex as the starting point and the base as the ending point using the operation part 62. For example, as shown in FIG. 2, the core axis determination part 33 determines the core axis 103 passing through the apex and the base and intersecting with the left ventricle 101.

### (Step S04)

The first image generator 34 generates SA view data in the short-axis cross-section orthogonally intersecting with the core axis based on the volume data showing the region of the heart. For example, as shown in FIG. 2, the first image generator 34 sets the plurality of short-axis cross-sections 111 in equal intervals in the region 110 between the starting point (the apex) and the ending point (the base) of the core axis 103. As an example, the first image generator 34 sets 40 frames of the short-axis cross-sections 111 in the region 110 and generates 40 frames of the SA view data.

### (Step S05)

The function calculator 4 calculates the difference in the lateral wall distance, which is one example of the functional information of the heart, based on each SA view data. Specifically, the function calculator 4 determines the difference in the lateral wall distance at the end diastole and the end systole. For example, the function calculator 4 determines the differences in the lateral wall distances at 72 locations at intervals of 5° for one short-axis cross-section 111 regarding 40 frames of the short-axis cross-sections 111. Alternatively, the function calculator 4 may calculate changes in the wall thickness. For example, the function calculator 4 calculates changes in the wall thickness at 72 locations at intervals of 5° for one short-axis cross-section 111 regarding 40 frames of the short-axis cross-sections 111.

### (Step S06)

The second identification part 32 receives the volume data showing the region of the heart from the first identification part 31, and identifies the calcified sites in the region of the heart from volume data based on pixel values such as CT values. For example, the second identification part 32 may identify the calcified sites from the volume data at the end diastole or may identify the calcified sites from the volume data at the end systole.

### (Step S07)

The thickness calculator 35 determines the thickness of the calcified sites in each short-axis cross-section based on the calcified sites identified by means of the second identification part 32 and the plurality of SA view data generated by means of the first image generator 34. For example, the thickness calculator 35 determines the thickness of the calcified sites in each short-axis cross-section at the end systole. As an example, the thickness calculator 35 determines the thickness at 72 locations for one short-axis cross-section 111 regarding 40 frames of the short-axis cross-sections 111.

Note that the sequence in which processing at Step S03 through Step S05 and processing at Step S06 may be executed in reverse order or may be executed simultaneously.

### (Step S08)

The bullseye map generator 51 converts the combination of the thickness of the calcified sites and the difference in the lateral wall distance into color and generates a bullseye map. For example, the bullseye map generator 51 determines the color corresponding to the combination of the thickness of the calcified sites and the difference in the lateral wall distance, using the color map 150 shown in FIG. 6. As an example, the bullseye map generator 51 determines the colors at 72 locations for each 5° interval for one short-axis cross-section 111 regarding 40 frames of the short-axis cross-sections 111. Moreover, the bullseye map generator 51 plots the color at each location (for example, at 72 locations) in the short-axis cross-section 111 at the apex on the inner most circle in the bullseye map and plots the color at each location (for example, at 72 locations) in the short-axis cross-section 111 at the base on the outermost circle in the bullseye map. Note that the bullseye map generator 51 may generate the bullseye map using the changes in wall thickness.

### (Step S09)

The second image generator 52 generates three-dimensional image data sterically showing the heart upon receiving volume data showing the region of the heart from the first identification part 31.

Note that the sequence in which processing at Step S03 through Step S08 as well as processing at Step S09 may be executed in reverse order or may be executed simultaneously.

### (Step S10)

For example, as shown in FIG. 6, the display controller 53 causes the display 61 to display a bullseye map 160. Moreover, as shown in FIG. 7, the display controller 53 may cause the display 61 to display the three-dimensional image 200. The display controller 53 may cause the display 61 to display the bullseye map 160 and the three-dimensional image 200 side by side.

If the first threshold is set with respect to the thickness of the calcified sites by the operator, the display controller 53 causes the display 61 to display the region surrounded by the frame 171 in the bullseye map 170 (the region in which the thickness of the calcified sites exceeds the first threshold). Moreover, if the second threshold with respect to the difference in the lateral wall distance (or changes in the wall thickness) is set by the operator, the display controller 53 causes the display 61 to display the frame 172 (the region in which the difference in the lateral wall distance (or the changes in the wall thickness) is smaller than the second threshold), on the bullseye map 170. Moreover, the display controller 53 may cause the display 61 to display the bullseye map 180 in which the critical region (the region 173) is displayed. As shown in FIG. 7, in the three-dimensional image 200, the display controller 53 may cause the display 61 to display the region 201 in which the thickness of the calcified sites exceeds the first threshold so as to be distinguishable. In the three-dimensional image 200, the display controller 53 may cause the display 61 to display the region 202 in which the difference in the lateral wall distance (or changes in the wall thickness) is smaller than the second threshold so as to be distinguishable. In the three-dimensional image 200, the display controller 53 may cause the display 61 to display the region 203 defined as the critical region so as to be distinguishable.

As above, the medical image processing apparatus 1 according to the first embodiment allows a combination of morphological information and functional information to be converted into a color, so as to generate the bullseye map using this color, allowing morphological information and functional information to be displayed in association with each other. That is, it will be possible to display the information of the sites affecting the movement of the heart muscle (for example, the calcified sites) and the information of the movement of the heart muscle into one bullseye map. Accordingly, it is possible for observers such as physicians to associate the calcified sites with the movement of the heart muscle and understand them by referring to the bullseye map. As a result, it becomes easy to examine the treatment plan.

Moreover, by associating the bullseye map with the three-dimensional image, it becomes possible for observers such as physicians to easily understand the position of the calcified sites by means of the three-dimensional image, while understanding the movement of the heart muscle by means of the bullseye map.

Moreover, by restricting the display region by means of the first threshold, it becomes possible for observers such as physicians to easily understand the region in which the calcified sites are thick. Moreover, by restricting the display region by means of the second threshold, it becomes possible for observers to easily understand the region in which the function of the heart muscle is deteriorating. Moreover, by restricting the display region by means of the first threshold and the second threshold, it becomes possible for observers to understand the region in which the calcified sites are thick and function of the heart muscle is deteriorating as the critical region.

Moreover, the medical imaging apparatus 90 may comprise functions of the medical image processing apparatus 1. In such cases, the medical imaging apparatus 90 generates volume data by capturing images of the heart and furthermore executes the function of the medical image processing apparatus 1. Accordingly, the medical imaging apparatus 90 generates the bullseye map in which morphological information and functional information are combined. Accordingly, even if the medical imaging apparatus 90 functions as the medical image processing apparatus 1, it is possible to obtain the same effect as the medical image processing apparatus 1.

### Second Embodiment

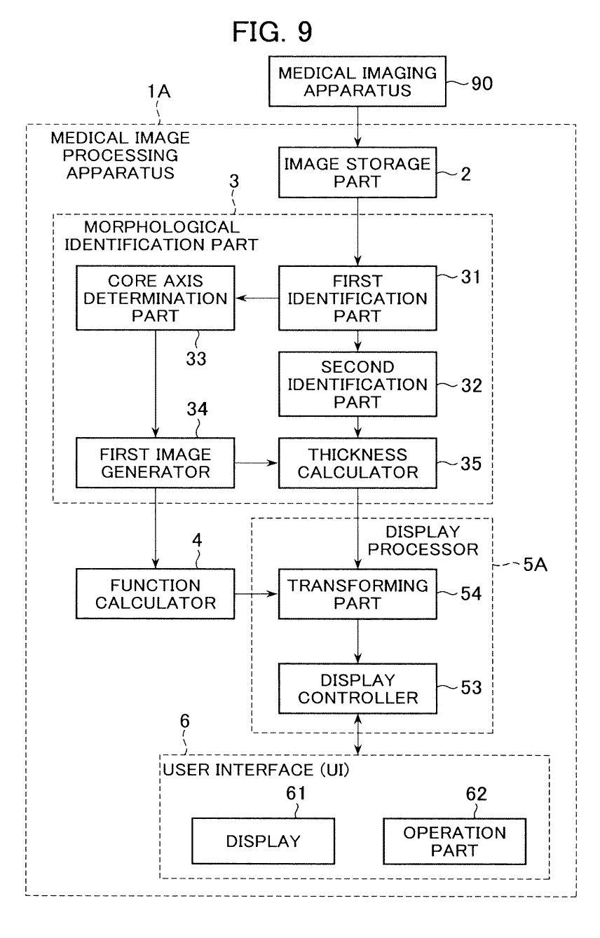

An explanation is provided with reference to FIG. 9 regarding the medical image processing apparatus according to the second embodiment. A medical image processing apparatus 1A according to the second embodiment comprises a display processor 5A in place of the display processor 5 according to the first embodiment. In the second embodiment, an explanation is provided for differences from the first embodiment. The medical image processing apparatus 1A according to the second embodiment adds the color corresponding to the combination of morphological information and functional information to the medical image to be displayed without generating a bullseye map.

### (Designating the Short-Axis Cross-Section)

As an example, an explanation is provided regarding cases of applying the color corresponding to the combination of morphological information and functional information to the short-axis image in the short-axis cross-section to be displayed. In such cases, the operator designates the position of any short-axis cross-section using the operation part 62. For example, as shown in FIG. 2, using the operation part 62, the operator designates the position of any short-axis cross-section 111 within the region 110 between the starting point (the apex) and the ending point (the base) of the core axis 103. The operator may designate the short-axis cross-section at any time phase, using the operation part 62. As an example, an explanation is provided for cases in which the short-axis cross-section is designated at the end diastole or at the end systole.

### (Morphological Identification Part 3)

The morphological identification part 3 determines the thickness of the calcified sites in the short-axis cross-section 111 designated by the operator. The first image generator 34 generates SA view data in the short-axis cross-section 111 designated by the operator. If the end diastole is designated, the first image generator 34 generates the SA view data at the end diastole based on the volume data at the end diastole. The thickness calculator 35 determines the thickness of the calcified sites in the short-axis cross-section 111 at the end diastole. If the end systole is designated, the first image generator 34 generates the SA view data at the end systole based on the volume data at the end systole. The thickness calculator 35 determines the thickness of the calcified sites in the short-axis cross-section 111 at the end systole. For example, as is the case with the first embodiment, the thickness calculator 35 determines the thickness of the calcified sites at 72 locations for each 5° interval, centered on the rotation of the core axis 103.

### (Function Calculator 4)

The function calculator 4 determines the difference in the lateral wall distance (changes in the wall thickness) in the short-axis cross-section 111 designated by the operator. For example, as is the case with the first embodiment, the function calculator 4 determines the difference in the lateral wall distance sites at 72 locations for each 5° interval, centered on the rotation of the core axis 103.

### (Display Processor 5A)

The display processor 5A comprises a display controller 53 and a transforming part 54. The display processor 5A includes the transforming part 54 in place of the bullseye map generator 51.

### (Transforming Part 54)

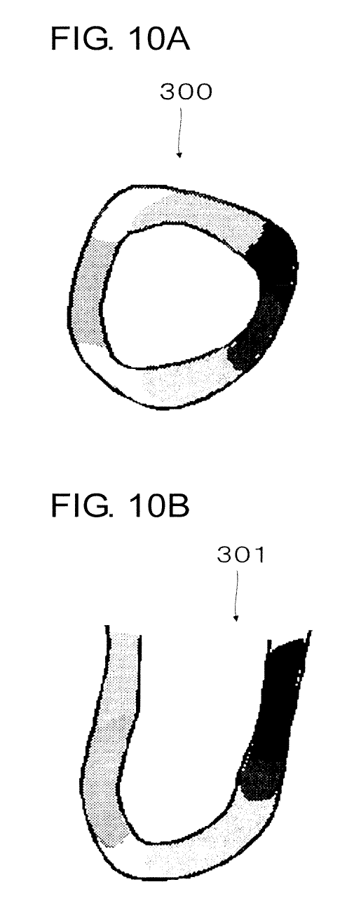

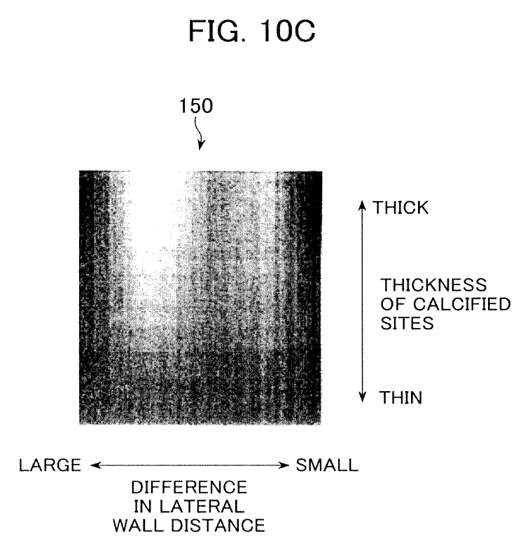

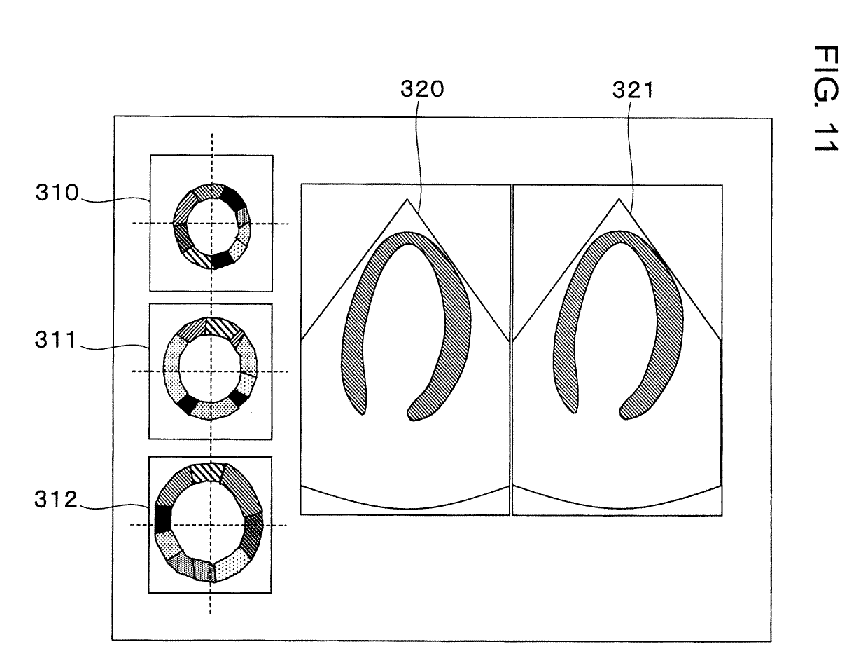

The transforming part 54 determines a color corresponding to a combination of morphological information and functional information, using the two-dimensional color map having two axes. That is, the transforming part 54 converts a combination of morphological information and functional information into a color. An example of the color map is shown in FIG. 10A to FIG. 10C. FIG. 10A is a diagram showing the short-axis image. FIG. 10B is a diagram showing the long-axis image. FIG. 10C is a diagram showing the color map. For example, as is the case with the first embodiment, the transforming part 54 uses the color map 150 shown in FIG. 10C.

As an example, the transforming part 54 determines the color corresponding to the combination of the thickness of the calcified sites and the difference in the lateral wall distance, using the color map 150. If the end diastole is designated, the transforming part 54 uses the thickness of the calcified sites at the end diastole as the thickness of the calcified sites. If the end systole is designated, the transforming part 54 uses the thickness of the calcified sites at the end systole as the thickness of the calcified sites. For example, the transforming part 54 determines the color corresponding to 72 locations for each 5° interval with respect to the designated short-axis cross-section 111. The transforming part 54 may determine the color using the changes in the wall thickness.

### (Display Controller 53)

The display controller 53 causes the display 61 to display the short-axis image based on the SA view data generated by means of the first image generator 34. The display controller 53 applies the color determined by means of the transforming part 54 to each site of the short-axis image to be displayed by the display 61. If the end diastole is designated, the display controller 53 causes the display 61 to display the short-axis image at the end diastole and applies the color to each site in the short-axis image to be displayed by the display 61. If the end systole is designated, the display controller 53 causes the display 61 to display the short-axis image at the end systole and applies the color to each site in the short-axis image to be displayed by the display 61. For example, as shown in FIG. 10A, the display controller 53 causes the display 61 to display a short-axis image 300 in which the color is applied to each section.

### (Specifications of the Long-Axis Cross-Section)

Moreover, the medical image processing apparatus 1A may apply the color corresponding to the combination of morphological information and functional information to the long-axis image in the long-axis cross-section orthogonally intersecting the short-axis cross-section to be displayed. In such cases, the operator specifies the position of any long-axis cross-section using the operation part 62. For example, the operator specifies the long-axis cross-section including the core axis 103 shown in FIG. 2, using the operation part 62. The operator may specify the long-axis cross-section at any time phase, using the operation part 62. As an example, an explanation is provided for cases in which the long-axis cross-section at the end diastole or the end systole is designated.

The morphological identification part 3 determines the thickness of the calcified site in the long-axis cross-section designated by the operator. The first image generator 34 generates long-axis view data in the long-axis cross-section designated by the operator. If the end diastole is designated, the first image generator 34 generates the long-axis view data at the end diastole based on the volume data at the end diastole. The thickness calculator 35 determines the thickness of the calcified sites in the long-axis cross-section at the end diastole. If the end systole is designated, the first image generator 34 generates the long-axis view data at the end systole based on the volume data at the end systole. The thickness calculator 35 determines the thickness of the calcified sites in the long-axis cross-section at the end systole. For example, the thickness calculator 35 determines the thickness of the calcified site at the plurality of locations for each interval set in advance.

The function calculator 4 determines the difference in the lateral wall distance (or changes in the wall thickness) in the long-axis cross-section designated by the operator. For example, the function calculator 4 determines the difference in the lateral wall distance (or changes in the wall thickness) at a plurality of locations for each interval set in advance.

The transforming part 54 determines the color corresponding to a combination of the thickness of the calcified sites and the difference in the lateral wall distance using the color map 150.

The display controller 53 causes the display 61 to display the long-axis image based on the long-axis view data generated by means of the first image generator 34. The display controller 53 applies the color determined by means of the transforming part 54 to each location of the long-axis image to be displayed by the display 61. If the end diastole is designated, the display controller 53 causes the display 61 to display the long-axis image at the end diastole and applies the color to each location of the long-axis image to be displayed by the display 61. If the end systole is designated, the display controller 53 causes the display 61 to display the long-axis image at the end systole and applies the color to each location of the long-axis image to be displayed by the display 61. For example, as shown in FIG. 10B, the display controller 53 causes the display 61 to display a long-axis image 301 in which the color is applied to each location.

The display controller 53 may cause the display 61 to display the short-axis image 300 in which the color is applied to each location and the long-axis image 301 in which the color is applied to each location side by side. The display controller 53 may cause the display 61 to display either one image of the short-axis image 300 or the long-axis image 301.

The medical image processing apparatus 1A may generate a MPR image in any cross-section at any time phase and applies the color to the MPR image to be displayed. Moreover, the medical image processing apparatus 1A may generate a three-dimensional image and apply the color to the three-dimensional image to be displayed.

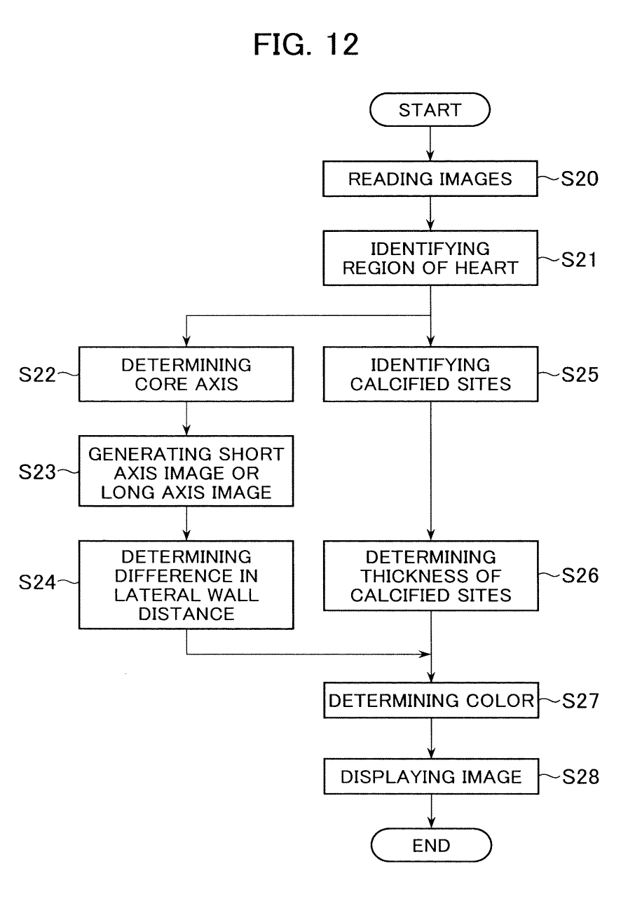

For example, the medical image processing apparatus 1A may generate a plurality of SA view data and a plurality of long-axis view data and display each image side by side. A display example of the plurality of images is shown in FIG. 11. FIG. 11 is a diagram showing a display example of the short-axis images and the long-axis images. For example, the medical image processing apparatus 1A generates a short-axis image 310, a short-axis image 311, and a short-axis image 312 with varying positions of the short-axis cross-section and displays each short-axis image with colors applied. Moreover, the medical image processing apparatus 1A generates a long-axis image 320 and a long-axis image 321 with varying positions of the long-axis cross-section and displays each long-axis image with colors applied. In this way, the plurality of images may be displayed side by side.

As is the case with the first embodiment, the display controller 53 may cause the display 61 to display the two-dimensional color map 150. Moreover, the display controller 53 may restrict the display region of the short-axis image or the long-axis image after setting the first threshold with respect to the thickness of the calcified site and the second threshold with respect to the functional information.

Note that the function of the transforming part 54 may be executed by a program. For example, a conversion program for executing the function of the transforming part 54 is stored in a storage device (not shown in the figures). As the processing device such as CPU executes the conversion program, the function of the transforming part 54 is executed.

### (Action)

An explanation is provided regarding actions of the medical image processing apparatus 1A according to the second embodiment, with reference to the flow chart shown in FIG. 12.

### (Step S20)

The first identification part 31 reads the plurality of volume data from the image storage part 2.

### (Step S21)

The first identification part 31 identifies the region of the heart from each volume data based on pixel values such as the CT value. For example, the first identification part 31 identifies the region of the heart from the volume data at the end diastole and identifies the region of the heart from the volume data at the end systole.

### (Step S22)

The core axis determination part 33 determines the core axis of the heart upon receiving volume data showing the region of the heart from the first identification part 31. For example, as shown in FIG. 2, the core axis determination part 33 determines the core axis 103 passing through the apex and the base and intersecting with the left ventricle 101.

### (Step S23)

The first image generator 34 generates SA view data in the short-axis cross-section orthogonally intersecting with the core axis based on the volume data showing the region of the heart. For example, the first image generator 34 generates the SA view data in the short-axis cross-section 111 designated by the operator. The first image generator 34 may generate the long-axis view data in the long-axis cross-section designated by the operator.

### (Step S24)

The function calculator 4 determines the difference in the lateral wall distance in the short-axis cross-section 111 designated by the operator. For example, the function calculator 4 determines the difference in the lateral wall distance at 72 locations at intervals of 5°. Alternatively, the function calculator 4 may calculate the changes in the wall thickness at 72 locations at intervals of 5° with respect to the short-axis cross-section 111. The function calculator 4 may calculate the difference in the lateral wall distance (or changes in the wall thickness) in the long-axis cross-section designated by the operator.

### (Step S25)

The second identification part 32 identifies the calcified sites in the region of the heart based on pixel values such as the CT value, upon receiving the volume data showing the region of the heart from the first identification part 31. For example, the second identification part 32 may identify the calcified sites from the volume data at the end diastole or it may identify the calcified sites from the volume data at the end systole.

### (Step S26)

The thickness calculator 35 determines the thickness of the calcified sites in the short-axis cross-section based on the calcified sites identified by means of the second identification part 32 and the SA view data generated by means of the first image generator 34. For example, the thickness calculator 35 determines the thickness of the calcified site in the short-axis cross-section at the end systole. As an example, the thickness calculator 35 determines the thickness at 72 locations. The thickness calculator 35 may calculate the thickness of the calcified sites in the long-axis cross-section based on the calcified sites identified by means of the second identification part 32 and the long-axis view data generated by means of the first image generator 34.

### (Step S27)

The transforming part 54 converts the combination of the thickness of the calcified sites and the difference in the lateral wall distance into colors. For example, the transforming part 54 determines the color corresponding to the combination of the thickness of the calcified sites and the difference in the lateral wall distance, using the color map 150 shown in FIG. 10C. As an example, the transforming part 54 determines the color at 72 locations for each 5° interval with respect to the short-axis cross-section 111 designated. Note that the transforming part 54 may determine the color using changes in the wall thickness.

Note that the sequence in which processing at Step S22 through Step S24 and processing at Step S25 may be executed in reverse order or may be executed simultaneously.

### (Step S28)

The display controller 53 causes the display 61 to display the short-axis image based on the SA view data generated by means of the first image generator 34. The display controller 53 causes the display 61 to apply the color determined by means of the transforming part 54 to each location of the short-axis image to be displayed. For example, as shown in FIG. 10A, the display controller 53 causes the display 61 to display the short-axis image 300 in which the color has been applied to each location.

The display controller 53 may cause the display 61 to display the long-axis image based on the long-axis view data generated by means of the first image generator 34. The display controller 53 causes the display 61 to apply the color determined by means of the transforming part 54 to each location of the long-axis image to be displayed. For example, as shown in FIG. 10B, the display controller 53 causes the display 61 to display the long-axis image 301 in which the color has been applied to each location.

The display controller 53 may cause the display 61 to display the short-axis image 300 and the long-axis image 301 side by side, or may cause the display 61 to display either image.

As above, the same effect as the medical image processing apparatus 1 according to the first embodiment can be obtained even by means of the medical image processing apparatus 1A according to the second embodiment. That is, by converting the combination of morphological information and function information into a color and displaying the color to be superimposed on the short-axis image or the long-axis image, it becomes possible to associate morphological information with functional information to be displayed. That is, it becomes possible to display the information of the calcified site and the information of the heart muscle movement to be superimposed on the short-axis image or the long-axis image. Accordingly, it becomes possible for observers such as physicians to associate the calcified sites with the heart muscle movement and understand them by referring to the short-axis image or the long-axis image. As a result, it becomes easy to examine the treatment plan.

Moreover, by displaying the short-axis image or the long-axis image as well as the three-dimensional image, it becomes possible for observers to more easily understand the position of the calcified sites.

Moreover, as is the case with the first embodiment, by restricting the display region using the first threshold and the second threshold, it becomes possible for observers to easily understand the region in which the calcified sites are thick, the region in which the function of the heart muscle deteriorates, or the critical region.

Note that the medical imaging apparatus 90 may comprise the function of the medical image processing apparatus 1A. In such cases, by capturing images of the heart, the medical imaging apparatus 90 generates volume data and furthermore executes the function of the medical image processing apparatus 1A. Accordingly, the medical imaging apparatus 90 add the color corresponding to the combination of morphological information and functional information to the medical image to be displayed. In this way, even if the medical imaging apparatus 90 executes the function of the medical image processing apparatus 1A, it is possible to obtain the same effect as the medical image processing apparatus 1A.

### Third Embodiment

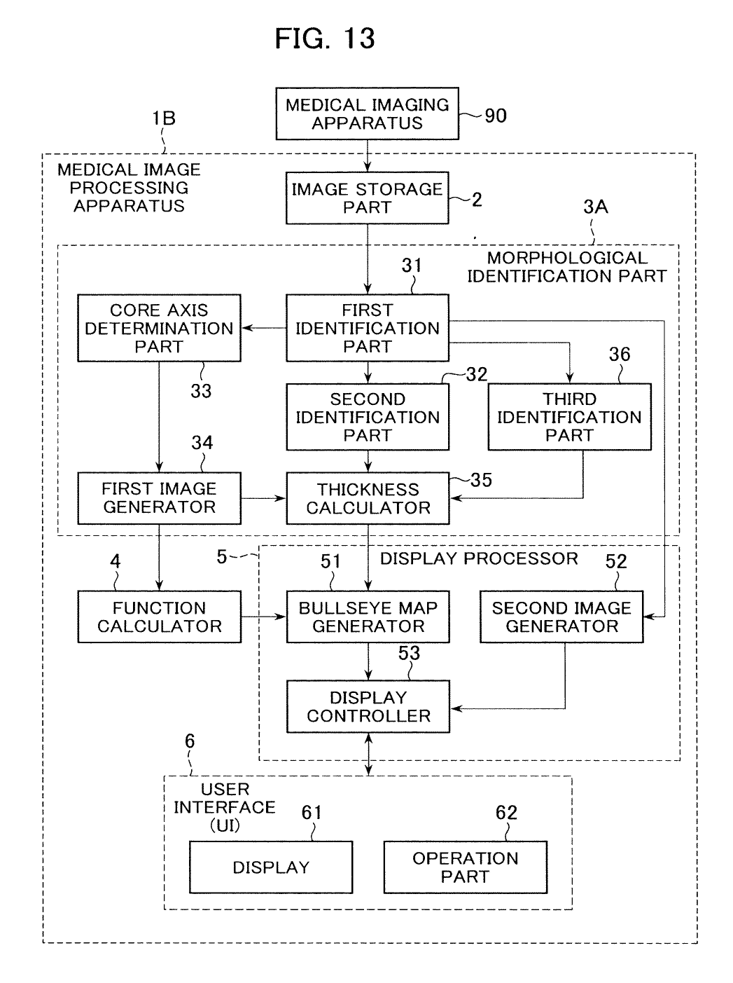

An explanation is provided regarding the medical image processing apparatus according to the third embodiment, with reference to FIG. 13. A medical image processing apparatus 113 according to the third embodiment comprises a morphological identification part 3A in place of the morphological identification part 3 according to the first embodiment. The third embodiment sets forth differences compared to the configuration according to the first embodiment. The medical image processing apparatus 1B according to the third embodiment determines the thickness of the calcified sites and the thickness of the fatty area as morphological information.

### (Morphological Identification Part 3A)

The morphological identification part 3A comprises a third identification part 36 in addition to the morphological identification part 3 according to the first embodiment. As is the case with the first embodiment, the morphological identification part 3A determines the thickness T of the calcified site regarding each short-axis cross-section 111 shown in FIG. 2. If 40 frames of the short-axis cross-sections 111 are set, the morphological identification part 3A determines the thickness T at 72 locations for each 5° interval for one short-axis cross-section 111 regarding 40 frames of the short-axis cross-sections 111. The morphological identification part 3A may calculate the thickness of the calcified sites at the end systole or it may calculate the thickness of the calcified sites at the end diastole.

### (Third Identification Part 36)

The third identification part 36 identifies the fatty area (myocardial fat) surrounding the heart from volume data upon receiving the volume data showing the region of the heart from the first identification part 31. For example, the third identification part 36 identifies the fatty area surrounding the heart based on pixel values such as the CT value. As an example, the third identification part 36 identifies the fatty area in the three-dimensional space, using the region growing method. For example, the third identification part 36 may identify the fatty area from the volume data at the end diastole or it may identify the fatty area from the volume data at the end systole. That is, the third identification part 36 may identify the fatty area at the end diastole or it may identify the fatty area at the end systole. Alternatively, the third identification part 36 may identify the fatty area from the volume data generated at each time phase within one heartbeat.

The thickness calculator 35 determines the thickness of the fatty area in each short-axis cross-section based on the fatty area identified by means of the third identification part 36 as well as the plurality of SA view data generated by means of the first image generator 34. For example, the thickness calculator 35 determines the thickness of the fatty area in each short-axis cross-section at the end systole. For example, as shown in FIG. 3B, the thickness calculator 35 determines the thickness of the fatty area by setting the direction from the core axis 103 toward the lateral wall 132 as the thickness. For example, the thickness calculator 35 determines the thickness of the fatty area at 72 locations for each 5° interval centered on the rotation of the core axis 103. The thickness calculator 35 determines the thickness of the fatty area regarding each short-axis cross-section at the end systole. If 40 frames of the short-axis cross-sections 111 are set, the thickness calculator 35 determines the thickness at 72 locations for one short-axis cross-section 111 regarding 40 frames of the short-axis cross-sections 111.

Alternatively, the thickness calculator 35 may calculate the thickness of the fatty area in each short-axis cross-section at the end diastole. In such cases, the thickness calculator 35 determines the thickness of the fatty area at 72 locations for each 5° interval regarding each short-axis cross-section at the end diastole.

### (Function Calculator 4)

As is the case with the first embodiment, the function calculator 4 determines the difference in the lateral wall distance (or changes in the wall thickness) for each location of each short-axis cross-section. For example, the function calculator 4 determines the difference in the lateral wall distance (or changes in the wall thickness) at 72 locations for each 5° interval centered on the rotation of the core axis 103.

### (Bullseye Map Generator 51)

As is the case with the first embodiment, the bullseye map generator 51 generates a bullseye map based on morphological information calculated by means of the morphological identification part 3A and functional information calculated by means of the function calculator 4. The bullseye map generator 51 according to the third embodiment generates the bullseye map based on the thickness of the calcified sites, the thickness of the fatty area, and the difference in the lateral wall distance (or changes in the wall thickness).

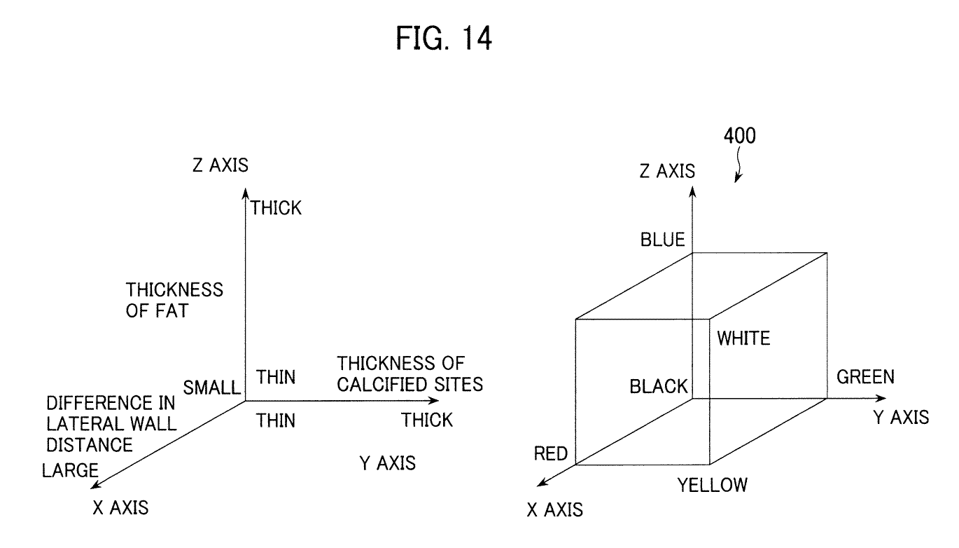

First, the bullseye map generator 51 determines the color corresponding to the combination of morphological information and functional information using the three-dimensional color map having three axes. An example of the color map is shown in FIG. 14. FIG. 14 is a diagram schematically showing the color map. For example, the bullseye map generator 51 uses the color map 400 shown in FIG. 14. The color map 400 has three axes (X axis, Y axis, and Z axis). The X axis corresponds to the difference in the lateral wall distance (or changes in the wall thickness). The Y axis corresponds to the thickness of the calcified sites. The Z axis corresponds to the thickness of the fatty area. The color map 400 shows, for example, the distribution of the combination of color phase and color saturation. That is, the color map 400 defines the combination of color phase and color saturation corresponding to the combination of the difference in the lateral wall distance (or changes in the wall thickness), the thickness of the calcified sites, and the thickness of the fatty area. For example, colors are applied on the color map 400 such that the larger the difference in the lateral wall distance (or changes in the wall thickness), the redder the color turns, and the smaller the difference in the lateral wall distance, the blacker the color turns. Moreover, the colors are applied on the color map 400 such that the thicker the calcified sites, the greener the color turns, and the thinner the calcified sites, the blacker the color turns. Moreover, the colors are applied on the color map 400 such that the thicker the fatty area, the bluer the color turns, and the thinner the fatty area, the blacker the color turns. The color map 400 is prepared in advance and stored in a storage (not shown in the figures).

As an example, the bullseye map generator 51 determines the color corresponding to the combination of the difference in the lateral wall distance, the thickness of the calcified site, and the thickness of the fatty area, using the color map 400. Specifically, the bullseye map generator 51 determines the coordinate of the X axis based on the difference in the lateral wall distance, determines the coordinate of the Y axis based on the thickness of the calcified site, and determines the coordinate of the Z axis based on the thickness of the fatty area. The bullseye map generator 51 identifies the color corresponding to the coordinate of the X axis, the coordinate of the Y axis, and the coordinate of the Z axis from the color map 400. As the thickness of the calcified sites and the thickness of the fatty area, the bullseye map generator 51 may use the thickness at the end systole or it may use the thickness at the end diastole.

The bullseye map generator 51 determines the color at each location in each short-axis cross-section. For example, the bullseye map generator 51 determines the color at 72 locations for one short-axis cross-section 111 regarding 40 frames of the short-axis cross-sections 111.

The bullseye map generator 51 generates a bullseye map using the color at each location of each short-axis cross-section 111. As is the case with the first embodiment, the bullseye map generator 51 allocates the color at each location (for example, at 72 locations) in the short-axis cross-section 111 at the apex on the inner most circle in the bullseye map, while allocating the color at each location (for example, at 72 locations) in the short-axis cross-section 111 at the base on the outermost circle in the bullseye map. For example, as shown in FIG. 5, the bullseye map generator 51 sets the apex as the center of the circle 140 and the base as the outermost side of the circle 140 and plots the color for 40 frames on each concentric circle regarding the color at 72 locations at intervals of 5° for one frame of the short-axis cross-section 111

As is the case with the first embodiment, the bullseye map generator 51 may generate the bullseye map using changes in the wall thickness. In such cases, the bullseye map generator 51 generates the bullseye map upon determining the color corresponding to the combination of changes in the wall thickness, the thickness of the calcified sites, and the thickness of the fatty area, using the color map 400.

The display controller 53 causes the display 61 to display the bullseye map generated by means of the bullseye map generator 51. The display controller 53 may cause the display 61 to display the bullseye map and the three-dimensional image of the heart side by side. The display controller 53 may cause the display 61 to display the bullseye map and the short-axis image or the long-axis image side by side.

As is the case with the first embodiment, the display controller 53 may cause the display 61 to display the three-dimensional color map 400. Moreover, the display controller 53 may restrict the display region of the bullseye map by setting the first threshold with respect to the thickness of the calcified sites, setting the second threshold with respect to the functional information, and setting the third threshold with respect to the thickness of the fatty area.

Note that the function of the third identification part 36 may be executed by a program. For example, a third identification program for executing the function of the third identification part 36 is stored in a storage device (not shown in the figures). As the processing device such as the CPU executes the third identification program, the function of the third identification part 36 is executed.

### (Action)

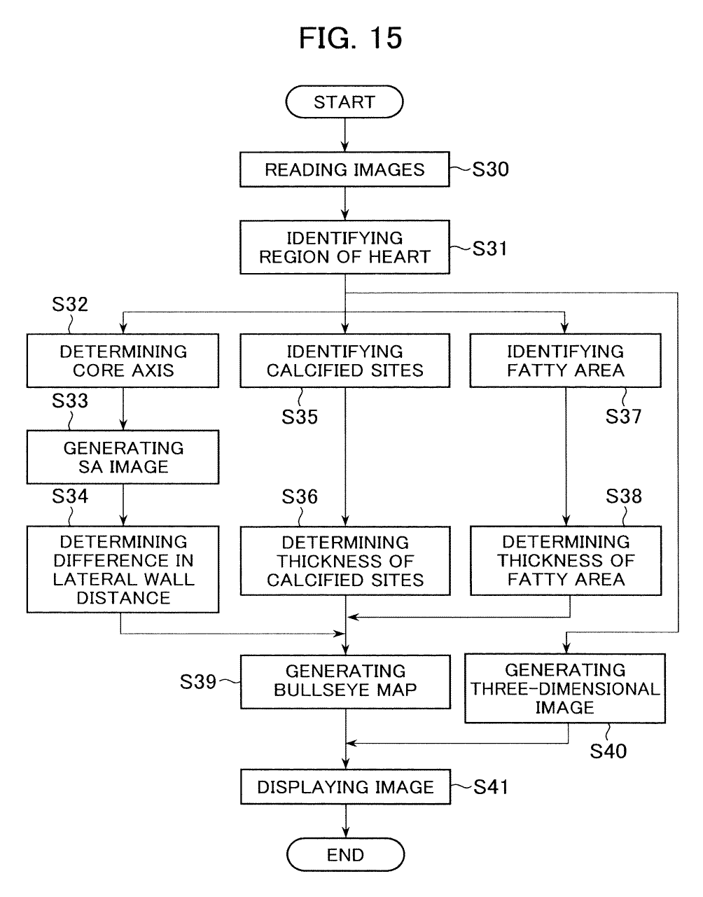

An explanation is provided regarding the action of the medical image processing apparatus 1B according to the third embodiment, with reference to the flow chart shown in FIG. 15.

### (Step S30)

The first identification part 31 reads the plurality of volume data from the image storage part 2.

### (Step S31)

The first identification part 31 identifies the region of the heart from each volume data based on pixel values such as the CT value. For example, the first identification part 31 identifies the region of the heart from the volume data at the end diastole and identifies the region of the heart from the volume data at the end systole.

### (Step S32)

The core axis determination part 33 determines the core axis of the heart upon receiving volume data showing the region of the heart from the first identification part 31. For example, the operator identifies the apex as the starting point and the base as the ending point, using the operation part 62. For example, as shown in FIG. 2, the core axis determination part 33 determines the core axis 103 passing through the apex and the base and intersecting with the left ventricle 101.

### (Step S33)

The first image generator 34 generates SA view data in the short-axis cross-section orthogonally intersecting with the core axis based on volume data showing the region of the heart. For example, as shown in FIG. 2, in the region 110 between the starting point (the apex) and the ending point (the base) of the core axis 103, the first image generator 34 sets 40 frames of the short-axis cross-sections 111 and generates 40 frames of the SA view data.

### (Step S34)

The function calculator 4 determines the difference in the lateral wall distance, which is one example of functional information of the heart, based on each SA view data. For example, the function calculator 4 determines the difference in the lateral wall distance at 72 locations at intervals of 5° for one short-axis cross-section 111 regarding 40 frames of the short-axis cross-sections 111. Alternatively, the function calculator 4 may calculate changes in the wall thickness. For example, the function calculator 4 determines changes in the wall thickness at 72 locations at intervals of 5° for one short-axis cross-section 111 regarding 40 frames of the short-axis cross-sections 111.

### (Step S35)

The second identification part 32 identifies calcified sites in the region of the heart from the volume data based on pixel values such as the CT value, upon receiving the volume data showing the region of the heart from the first identification part 31. For example, the second identification part 32 may identify the calcified sites from the volume data at the end diastole or it may identify the calcified sites from the volume data at the end systole.

### (Step S36)

The thickness calculator 35 determines the thickness of the calcified sites in each short-axis cross-section based on the calcified sites identified by means of the second identification part 32 and the plurality of SA view data generated by means of the first image generator 34. For example, the thickness calculator 35 determines the thickness of the calcified sites in each short-axis cross-section at the end systole. As an example, the thickness calculator 35 determines the thickness at 72 locations for one short-axis cross-section 111 regarding 40 frames of the short-axis cross-sections 111.

### (Step S37)

The third identification part 36 identifies the fatty area surrounding the heart based on pixel values such as the CT value, upon receiving volume data showing the region of the heart from the first identification part 31. For example, the third identification part 36 may identify the fatty area from the volume data at the end diastole or it may identify the fatty area from the volume data at the end systole.

### (Step S38)

The thickness calculator 35 determines the thickness of the fatty area in each short-axis cross-section based on the fatty area identified by means of the third identification part 36 and the plurality of SA view data generated by means of the first image generator 34. For example, the thickness calculator 35 determines the thickness of the fatty area in each short-axis cross-section at the end systole. As an example, the thickness calculator 35 determines the thickness at 72 locations for one short-axis cross-section 111 regarding 40 frames of the short-axis cross-sections 111.

Note that the sequence in which processing at Step S32 through Step S33, processing at Step S35 and processing at Step S37 may be executed in reverse order or may be executed simultaneously.

### (Step S39)

The bullseye map generator 51 converts the combination of the thickness of the calcified sites, the thickness of the fatty area, and the thickness of the lateral wall distance into colors and generates a bullseye map. For example, the bullseye map generator 51 determines the color corresponding to the combination of the thickness of the calcified sites, the thickness of the fatty area, and the thickness of the lateral wall distance, using the color map 400 shown in FIG. 14. As an example, the bullseye map generator 51 determines the color at 72 locations for each 5° interval for one short-axis cross-section 111 regarding 40 frames of the short-axis cross-sections 111. Moreover, the bullseye map generator 51 plots the color at each location (for example, at 72 locations) in the short-axis cross-section 111 at the apex on the inner most circle in the bullseye map and plots the color at each location (for example, at 72 locations) in the short-axis cross-section 111 at the base on the outermost circle in the bullseye map. Note that the bullseye map generator 51 may generate the bullseye map using the changes in the wall thickness.

### (Step S40)

The second image generator 52 generates three-dimensional image data sterically showing the heart upon receiving the volume data showing the region of the heart from the first identification part 31.

Note that the sequence in which processing at Step S32 through Step S39 and processing at Step S40 may be executed in reverse order or may be executed simultaneously.

### (Step S41)

The display controller 53 causes the display 61 to display the bullseye map. Moreover, the display controller 53 may cause the display 61 to display a three-dimensional image. The display controller 53 may cause the display 61 to display the bullseye map and the three-dimensional image side by side.

As above, the medical image processing apparatus 1B according to the third embodiment can achieve the same effect as the medical image processing apparatus 1 according to the first embodiment. That is, it becomes possible to show information of the calcified sites, information of the heart muscle movement, and information of the fatty area on one bullseye map. It is believed that there is a risk that fat surrounding the heart promotes calcification. Usage of the medical image processing apparatus 1B according to the third embodiment allows generating a bullseye map including information of the fatty area. In this way, by adding the information of the fatty area to the information of the calcified sites and the information of the heart muscle movement and displaying them on the bullseye map, it becomes possible for observers to associate the calcified sites, the heart muscle movement, and the fatty area with each other and understand them.

Moreover, by associating the bullseye map with the three-dimensional image, it becomes possible for observers to easily understand the heart muscle movement, the position of the calcified sites, and the position of the fatty area.

Moreover, as is the case with the first embodiment, by restricting the display region using the first threshold, second threshold, and third threshold, it is possible for observers to easily understand the region in which the calcified sites are thick, the regions in which the function of the heart muscle is deteriorating, the regions in which fat is thick, and the critical region.

Besides the medical image processing apparatus according to the above first embodiment through the third embodiment, processing according to the first embodiment may be combined with processing according to the second embodiment. That is, the bullseye map may be generated, and furthermore, a short-axis image or long-axis image may be generated. Moreover, processing according to the third embodiment may be applied to processing according to the second embodiment. That is, colors corresponding to the combination of the thickness of the calcified sites, the thickness of the fatty area, and the difference in the lateral wall distance (or changes in the wall thickness) may be determined, the colors may be applied to the short-axis image or the long-axis image, to be displayed.

In the above first embodiment, second embodiment, and third embodiment, the morphological identification part 3 may determine the thickness of a myocardial infarction as morphological information. The morphological identification part 3 reads the volume data obtained by means of contrast imaging from the image storage part 2 and identifies the myocardial infarction based on pixel values such as the CT value. As is the case with the above processing, the morphological identification part 3 determines the thickness of myocardial infarctions at each location of each short-axis cross-section. For example, the morphological identification part 3 determines the thickness of the myocardial infarction at 72 locations for each 5° interval regarding 40 frames of the short-axis cross-sections, while the display processor 5 generates the bullseye map by setting the thickness of the myocardial infarction as the morphological information and causes the display 61 to display this bullseye map. Alternatively, the display processor 5 may set the thickness of the myocardial infarction as morphological information, apply colors to the short-axis image and cause the display 61 to display them. Alternatively, the morphological identification part 3 may determine the thickness of the myocardial infarction at each location of the long-axis cross-section. In such cases, the display processor 5 sets the thickness of the myocardial infarction as morphological information, applies colors to the long-axis image and causes the display 61 to displays them. In this way, even if the thickness of the myocardial infarction is used as morphological information, as is the case with the above first embodiment through the third embodiment, it becomes possible for observers to associate morphological information with functional information and understand them.

Moreover, in the above first embodiment, second embodiment, and third embodiment, the function calculator 4 may calculate blood flow in the capillaries of the myocardial tissue or the corresponding vascular function system (for example, coronary artery) as functional information. An explanation is provided below regarding an example in which the blood flow in the coronary artery is calculated; however, the same is applicable even to other blood vessels. The morphological identification part 3 reads the plurality of volume data obtained by means of contrast imaging from the image storage part 2 and identifies the coronary artery based on pixel values such as the CT value. The function calculator 4 determines the capacity of the blood passing through in unit time at each location of the coronary artery shown respectively in each short-axis cross-section. For example, the function calculator 4 determines the amount of the blood flow in the coronary artery regarding 40 frames of the short-axis cross-sections. The display processor 5 generates a bullseye map by setting the amount of the blood flow in the coronary artery as functional information and causes the display 61 to display this bullseye map. Alternatively, the display processor 5 may apply colors to the short-axis image by setting the amount of the blood flow in the coronary artery as functional information and causes the display 61 to display them. Alternatively, the function calculator 4 may determine the amount of the blood flow in the coronary artery at each location of the long-axis cross-section. In such cases, the display processor 5 applies colors to the long-axis image by setting the amount of the blood flow in the coronary artery as functional information and causes the display 61 to display them. In this way, even if the amount of the blood flow in the coronary artery is used as functional information, as is the case with the above first embodiment through the third embodiment, it becomes possible for observers to associate morphological information with functional information and understand them.

Moreover, in the above first embodiment, second embodiment, and third embodiment, the function calculator 4 may calculate changes in the ventricular volume ratio as functional information. In such cases, as is the case with the first embodiment, second embodiment, and third embodiment, the morphological identification part 3 identifies the thickness of the calcified sites of the heart or the fatty area surrounding the heart. As is the case with the first embodiment, second embodiment, and third embodiment, the function calculator 4 determines the inner wall distance at the end systole and the end diastole regarding each location of each short-axis cross-section.

For example, the function calculator 4 determines the inner wall distance α at the end systole and the inner wall distance β at the end diastole at 72 locations for each 5° interval centered on the rotation of the core axis 103 and determines the ventricular volume ratio by means of the following formula (1).

{(β²−α²)/β²}×100 [Formula 1]

The display processor 5 generates a bullseye map by setting the changes in the ventricular volume ratio as functional information and causes the display 61 to display the bullseye map. Alternatively, the display processor 5 may apply colors to the short-axis image by setting the changes in the ventricular volume ratio as functional information and cause the display 61 to display them. In this way, even if the changes in the ventricular volume ratio are used as functional information, as is the case with the above first embodiment through the third embodiment, it becomes possible for observers to associate morphological information with functional information and understand them.

Moreover, in the first embodiment and the third embodiment, instead of generating a bullseye map, the display processor 5 may apply colors corresponding to the combination of morphological information and functional information to a model diagram illustrating the anatomical organization of the subject and display it. For example, anatomical drawing data showing the anatomical drawing (schema) of the human body is stored in storage (not shown in the figures) in advance. As an example, the anatomical drawing data of the schema showing the heart is stored in the storage (not shown in the figures). As is the case with the above embodiments, the display processor 5 determines colors corresponding to the combination of morphological information and functional information. Moreover, the display processor 5 applies colors to each location of the schema of the heart and causes the display 61 to display them. In this way, even if the anatomical drawing of the human body is used, it becomes possible for observers to associate morphological information with functional information and understand them. Note that if the schema is displayed, the bullseye map generator 51 may not be provided in the display processor 5.

According to the first embodiment through the third embodiment described above, by associating morphological information with functional information to be displayed, it becomes possible for observers such as physicians to easily diagnose the disease.

A number of embodiments of the present invention were described; however, these embodiments provide examples and are not intended to limit the scope of the invention. It is possible that these novel embodiments may be embodied in other various forms, with various omissions, replacements, and changes able to be carried out without departing from the scope of the invention. These embodiments and the transformation thereof are included in the scope and subject matter of the invention, while simultaneously being included in the invention according to the claims and the equal scope thereof.

### EXPLANATION OF THE SYMBOLS

1, 1A, 1B Medical image processing apparatus
2 Image storage part
3, 3A Morphological identification part
4 Function calculator
5, 5A Display processor
6 User interface (UI)
31 First identification part
32 Second identification part
33 Core axis determination part
34 First image generator
35 Thickness calculator
36 Third identification part
51 Bullseye map generator
52 Second image generator
53 Display controller
54 Transforming part
61 Display
62 Operation part
90 Medical imaging apparatus

1: A medical image processing apparatus comprising:
a morphological identification part configured to identify morphological information related to thickness of a heart muscle of a subject or thickness of surrounding sites thereof from medical image data obtained by capturing an image of the subject by means of a medical imaging apparatus;
a function calculator configured to calculate cardiac function information related to movement of the heart muscle of the subject based on the medical image data; and
a display processor configured to cause a display to display a combination of the identified morphological information and the calculated cardiac function information with identification of colors.

2: The medical image processing apparatus according to claim 1, wherein:
the identified morphological information comprises information related to thickness of calcified sites of the heart muscle, thickness of myocardial fat, and thickness of myocardial infarcted sites.

3: The medical image processing apparatus according to claim 1, wherein:
the identified morphological information comprises information related to any one of changes in wall motion or the thickness of the heart muscle, blood flow in capillary of myocardial tissue or the corresponding vascular function system, or changes in ventricular volume ratio.

4: The medical image processing apparatus according to claim 1, wherein:
the display processor is configured to cause the display to display a combination of the thickness of the calcified sites of the heart muscle as the identified morphological information, and information related to the movement of the heart muscle as the calculated cardiac function information with identification of colors, as associated with a position of the heart muscle in a short-axis cross-section of the heart of the subject.

5: The medical image processing apparatus according to claim 1, wherein:
the display processor is configured to cause the display to display a combination of thickness of myocardial infarcted sites as the identified morphological information, and information related to blood flow in capillaries of the myocardial tissue or the corresponding vascular function system as the calculated cardiac function information with identification of colors, as associated with a position of the heart muscle in a short-axis cross-section of the heart of the subject.

6: The medical image processing apparatus according to claim 2, wherein:
the display processor is configured to cause the display to display medical images based on the medical image data and to display the medical images by applying colors corresponding to a combination of the identified morphological information and the calculated cardiac function information.

7: The medical image processing apparatus according to claim 2, wherein:
the display processor is configured to cause the display to display a model diagram illustratively representing an anatomical organization of the subject and to display the model diagram by applying colors corresponding to a combination of the identified morphological information and the calculated cardiac function information.

8: The medical image processing apparatus according to claim 1, wherein:
the morphological identification part is configured to identify the morphological information in each of a plurality of short-axis cross-sections that cross a core axis extending from a base to apex of the heart,
the function calculator is configured to calculate the cardiac function information in each of the plurality of the short-axis cross-sections; and
the display processor is configured to cause the display to display a bullseye map arranging colors corresponding to the combination of the identified morphological information and the calculated cardiac function information in a concentric manner according to positions between the base and the apex, with respect to each of the plurality of short-axis cross-sections.

9. The medical image processing apparatus according to claim 1, wherein:
the display processor is configured to cause the display to display colors with the thickness exceeding a first threshold which is set in advance.

10. The medical image processing apparatus according to claim 3, wherein:
the display processor is configured to cause the display to distinguishably display colors corresponding to a range smaller than a second threshold previously set, the range including information of changes in the wall motion or the thickness of the heart muscle, the blood flow in the capillary of the myocardial tissue or the corresponding vascular function system, or changes in the ventricular volume ratio.

11. The medical image processing apparatus according to claim 1, wherein:
the display processor causes the display to display a color map showing a relationship between the identified morphological information and the calculated cardiac function information.

12. A medical imaging apparatus comprising:
a morphological identification part configured to identify morphological information related to thickness of a heart muscle of a subject or thickness of surrounding sites thereof from medical image data obtained by capturing an image of the subject;
a function calculator configured to calculate cardiac function information related to movement of the heart muscle of the subject based on the medical image data; and
a display processor configured to cause a display to display a combination of the identified morphological information and the calculated cardiac function information which is identified by color.

13. A medical image processing program that causes a computer to execute:
a morphological identification function that identifies morphological information related to thickness of the heart muscle of the subject or thickness of surrounding sites thereof, upon receiving medical image data obtained by capturing an image of the subject;
a function calculation function that determines cardiac function information related to the heart muscle movement of the subject based on the medical image data; and
a display processing function that causes a display to display a combination of the identified morphological information and the calculated cardiac function information which is identified by color.
